(19) United States
(12) Patent Application Publication
(10) Pub. No.: US 2026/0083313 A1
(43) Pub. Date: Mar. 26, 2026

(54) Split Overtube to Prevent Looping During Colonoscopy

(71) Applicant: Rafal Sadurski, Greenwood, SC (US)

(72) Inventor: Rafal Sadurski, Greenwood, SC (US)

(21) Appl. No.: 18/893,733

(22) Filed: Sep. 23, 2024

(51) Int. Cl. A61B 1/31; A61B 1/00

(52) U.S. Cl. CPC A61B 1/31; A61B 1/00135

(57) ABSTRACT

The invention is a device that improves safety and efficiency of colonoscopy by preventing loop formation. It is the plastic overtube with the longitudinal split. The split allows the placement over the shaft of the endoscope when colonoscopy is already in progress and a loop is formed that hinders the further endoscope insertion. The overtube is segmented by oval openings of the split to facilitate placement and to allow using optimal lengths of the overtube. The fastening mechanism is used to close and secure the overtube on the shaft of the endoscope and then it is pushed inside the colon by sliding over the endoscope. Once in place, the overtube is kept steady and provides the semi-rigid conduit for further endoscope insertion. This conduit permits the axial force exerted by the operator to be translated to forward movement of the endoscope tip and eliminates dangerous loop formation.

### BACKGROUND OF THE INVENTION

A detailed overview of the colonoscopy procedure is presented in FIG. 1. A colonoscopy is a medical procedure in which a long, flexible tubular device equipped with a light and camera at its tip is introduced into the colon to visualize its interior. This device, known as an endoscope, is designed to navigate the length of the colon, allowing the operator to assess its condition and, if needed, perform therapeutic intervention.

The endoscope features a long shaft 10 connected to a handle 11, where control wheels 17 are located. These control wheels are linked to the tip of the endoscope via internal cables 12, enabling the operator to change the direction of the tip. This capability is crucial for maneuvering the endoscope through the bends of the colon 13 as the shaft is advanced from outside of the body. The handle of the endoscope is also connected to a light source processor 14 and a monitor 15 via a connector tube 16. The light illuminates the interior of the colon, while the camera transmits real-time images to the monitor, allowing the operator 18 to view the inside of the colon lumen on the monitor.

The endoscope operator 18 moves the control wheels 17 on the handle 11 with the left hand which steers the tip of the endoscope, while simultaneously inserting the shaft 10 into the body with their right hand. This coordinated effort enables the endoscope to navigate the lengthy and often winding structure of the colon. The procedure allows for a thorough examination of the colon's health, the collection of biopsy samples, and the performance of therapeutic interventions, such as the removal of polyps or the placement of sutures or clips and others. It is vital to evaluate the entire length of the colon during a colonoscopy to ensure a comprehensive assessment and to address any potential health issues effectively.

One of the difficulties that the operator may encounter during colonoscopy is looping of the endoscope which can prolong and complicate the procedure. The example of looping of the endoscope during colonoscopy is illustrated in FIGS. 2A and 2B. As the endoscope is introduced deeper into the lumen of the colon, it must navigate around the anatomical corners and bends 20. The colon walls provide resistance to the advancing shaft of the endoscope, allowing the distal end 21 to progress deeper into the colon. However, the anatomy of the colon is often very convoluted and as the endoscope is advanced, it can stretch parts of the colon and force it into unnatural shapes. When the operator attempts 22 to traverse tight corners, the endoscope may buckle, forming a “loop” 23.

The looping is a frequent event during colonoscopy, with estimates in the literature suggesting its occurrence in the majority of procedures—estimates range from 73% to 91% (References 1 and 2). Two parts of the colon—the sigmoid and transverse colon—are particularly susceptible to loop formation. These sections are suspended on the mesentery, making them more flexible and prone to looping.

FIGS. 3A, 3B, 3C, 3D, 3E illustrate the most commonly formed loops; their names originate from shapes the loop creates: N loop in the sigmoid colon 30, Alpha loop in the sigmoid colon 31, reverse Alpha loop in the sigmoid colon 32, simple loop in the transverse colon 33, Gamma loop in the transverse colon 34.

In most cases the operator can adjust the insertion and apply techniques that allow the endoscope to pass through curves without major difficulty. However, in about 10-20% of cases, the endoscope continues to recreate loops despite repeated attempts to navigate past them. This not only prolongs the procedure but also necessitates higher doses of sedation due to the increased discomfort for the patient. In smaller percentages of cases (estimated 3-5%) the looping may lead to inability to complete visualization of the entire colon.

The formation of loops can exert excessive pressure on the intestinal wall, leading to complications such as pain, cardiac arrhythmia due to vasovagal reflex, and, in severe cases, major bleeding and perforation of the colonic wall. Extensive literature is available regarding possible complications of colonoscopy (References 3, 4 and 5). Understanding and managing these loops is crucial for the successful completion of a colonoscopy, minimizing patient discomfort, and avoiding serious complications.

There are methods and devices designed to minimize looping. FIGS. 4A, 4B, and 4C. Illustrate one of such methods. The endoscope operator can identify the formation of a loop 40 when, despite advancing the shaft of the scope into the body 41, the distal end fails to move forward and may even paradoxically move backward. The operator partially withdraws the endoscope 42 while applying directional torque 43 to help straighten the scope. This allows the sigmoid colon to pleat along the shaft, reducing tension. The endoscope can then be reintroduced 44 often with the application of clockwise torque 45. This guides the endoscope through the curves of the colon without forming a loop. Another method to minimize the risk of loop formation is “splinting.” In this technique, the assistant applies pressure to the patient's abdominal wall to stabilize the intestines and limit their movement as the endoscope is advanced 46.

Additionally, positioning the patient differently—such as shifting them from the typical left side position to a supine (on the back) or prone (on the belly) position—can also reduce the likelihood of loop formation. While loop formation is often evident to the operator, determining the exact location and type of loop can be challenging. Therefore, the maneuvers of endoscope manipulation, splinting, and repositioning are often applied on a trial-and-error basis. Despite these interventions, there are instances where the endoscope cannot be advanced to the end of the colon, with an estimated 3-5% of colonoscopies failing to be completed due to looping.

Risk factors for difficult colonoscopies include female gender, advanced age, low body weight, and previous abdominal surgery. When a colonoscopy cannot be completed, alternative strategies to visualize the entire colon may include repeating the colonoscopy with a different physician, using specialized radiographic studies such as a barium enema or CT scan, or even considering surgery. In some cases, fluoroscopy (X-ray) or magnetic ring devices can be used to visualize loops more clearly. However, these tools require additional equipment, add complexity to the procedure and are not routinely employed due to their cumbersome nature. In addition to these techniques, other methods can be employed to manage and prevent looping during colonoscopy. These include the use of mechanical aids such as overtubes, advanced colonoscope designs, fluoroscopic guidance (Reference 6, 7 and 8).

The overtube is a hollow plastic tube that can be positioned over the endoscope by means of sliding it at the endoscope distal end. It can then advance inside the body to create a more rigid pathway for the endoscope. Some overtubes are designed with adjustable rigidity, allowing the operator to modify their stiffness as needed during the procedure. However, there are significant limitations with the currently available overtubes. One major issue is that the overtube must be positioned on the endoscope before the procedure begins. Since severe, repeated looping occurs only in a small percentage of cases, placing an overtube preemptively for every colonoscopy is impractical. Additionally, when an overtube is in place, it reduces the length of the endoscope shaft accessible to the operator, limiting maneuverability and control.

Due to these drawbacks, the use of overtubes to alter the flexibility of the endoscope is infrequent in colonoscopy. The overtube's practical limitations often outweigh its potential benefits in preventing or managing looping, particularly in routine procedures. The most common use of overtubes is during endoscopy of the stomach, particularly when removing foreign bodies. In such cases, multiple passages of the scope may be required, and the overtube helps protect the throat and esophagus from being scratched or injured by repeated insertions of the endoscope. Overtubes can also be used to provide additional channels through which biopsy forceps, retrieval devices, or other instruments can be introduced as needed. This can be particularly useful in complex procedures where multiple tools need to be employed simultaneously.

FIGS. 5, 6A, 6B, 7, 8A, 8B, 8C, and 8D illustrate the nature of our invention and its method of use. The invention presented here is an overube—plastic tube to be placed on the endoscope in order to provide conduit for it and prevent loop formation during colonoscopy. It substantially differs from currently available overtubes by having a longitudinal split 50 throughout its length. This feature allows the placement of the overtube on the endoscope while the procedure is already in progress and only the shaft, but not tip of the endoscope, is accessible outside of the patient's body. Since only a small fraction of the procedures requires extensive efforts to combat loop formation as needed, use of split overtube is cost-saving. The other feature is presence of the oval openings 51 along the split This separates the tube into segments permitting only partial length of it to be used for insertion. The remaining part of the overtube is bent away 71 and does not limit the shaft of the endoscope accessible for the operator. After a segment of the overtube split is spread out and the part of the shaft of the endoscope is introduced inside its lumen—FIG. 8A detail 80, the overtube is fastened using a closing mechanism. FIGS. 6 A and 6 B show the first embodiment of such a mechanism, FIG. 6C shows another alternative of the closing mechanism. While keeping the endoscope steady the overtube is then introduced inside the colon by means of sliding it on the endoscope. Variable number of segments are introduced in the same fashion depending on the need—FIG. 8B. Overtube is then bent on the site of the opening closest to the body and kept in a steady position. Because of the opening there is negligible increase in resistance between endoscope and overtube and endoscope can be operated in usual manner. Due to relative simplicity of the overtube (no additional attachments or complicated internal structure) it is expected to be less expensive to produce than currently available rigidized overtubes.

The overtube other feature is taper 55 and rounded edge 56 on the distal end of the overtube. allowing enhanced overtube safety and facilitating introduction. The proximal edge of the overtube is flared up 53 making it safer for use by preventing accidental introduction of the entire tube into the colon.

Patent citations

| | | | |
|---|---|---|---|
| U.S. Pat. No. 9,480,390 | Nov. 1, 2016 | Ashkan Farhadi | Endoscope Accessory |
| U.S. Pat. No. 9,125,560 | Sep. 15, 2015 | OLYMPUS CORPORATION, | Endoscope shape |
| | | Tokyo (JP) | detecting apparatus |
| U.S. Pat. No. 8,573,226 | Nov. 5, 2013 | USGI Medical Inc. | Apparatus and methods |
| | | | for performing transluminal |
| | | | gastrointestinal procedures |
| U.S. Pat. No. 10,314,663 | Jun. 11, 2019 | OLYMPUS CORPORATION, | Medical overtube |
| | | Tokyo (JP) | |
| U.S. Pat. No. 11,135,398 | Oct. 5, 2021 | Neptune Medical Inc. | Dynamically rigidizing |
| | | | composite medical |
| | | | structures |

### REFERENCES—NON PATENT CITATIONS

1) Colon Anatomy Based on CT Colonography and Fluoroscopy: Impact on Looping, Straightening and Ancillary Maneuvers in Colonoscopy. By Eickhoff at al. in Digestive and Liver Disease. 2010; 42 (4): 291-6.
2) Magnetic Imaging of Colonoscopy: An Audit of Looping, Accuracy and Ancillary Maneuvers. Shah S G, et al. Gastrointestinal Endoscopy. 2000; 52 (1): 1-8.
3) Complications of Colonoscopy. Fisher D A, et al. Gastrointestinal Endoscopy. 2011; 74 (4): 745-52.
4) Screening for Colorectal Cancer: U.S. Preventive Services Task Force Recommendation Statement. Annals of Internal Medicine. 2008; 149 (9): 627-37.
5) Incidence and Causes of Colonoscopic Perforations: A Single-Center Case Series. Loffeld R J, et al. Endoscopy. 2011; 43 (3): 240-2.
6) Going for the Loop: A Unique Overtube for the Difficult Colonoscopy. Hawari R, Pasricha P J. Journal of Clinical Gastroenterology. 2007; 41 (2): 138-40,
7) Novel Rigidizing Overtube for Colonoscope Stabilization and Loop Prevention (With Video). Wei M T et al. Gastrointestinal Endoscopy. 2021; 93 (3): 740-749.,
8) Overview of Upcoming Advances in Colonoscopy. Cheng W B et al. Digestive Endoscopy: Official Journal of the Japan Gastroenterological Endoscopy Society. 2012; 24 (1): 1-6.

### SUMMARY

The nature of this invention is a longitudinal split along the overtube that allows it to be placed on the endoscope while the colonoscopy is already in progress. This permits the use of the overtube only as needed—for example when the endoscope distorts the intestine with loop formation and other methods of eliminating the loop fail; this has been occurring only in about 3-5% of cases.

The second important feature of the invention are oval openings along the split. This facilitates insertion and control of the endoscope when inside the overtube. It also permits the use of variable length of the overtube without decreasing the length of the endoscope available for the operator outside the colon. Before the overtube placement the endoscope is partially withdrawn with torque straightening the endoscope. After the overtube is placed on the part of the shaft of the endoscope into the overtube, each segment is closed and secured with the fastening mechanism.

Once the desirable length of the overtube is introduced inside the colon by sliding over an endoscope, the overtube is bent at the site of entry into the body and kept steady there. Subsequently the endoscope is pushed further through the segments of the overtube inside the colon. The overtube provides semirigid conduit for endoscope where axial forces exerted by the operator can be better transferred to forward motion of the tip rather than being dissipated into bowing of the shaft and loop formation. This permits the endoscope with an overtube on part of its shaft to be more easily and faster inserted into the colon without being hindered by the loop formation.

### BRIEF DESCRIPTION OF THE DRAWINGS

In the drawings, closely related figures have the same number but different alphabetic suffices.

FIG. 1. Schematic representation of the colonoscopy procedure.

FIG. 2 A. The endoscope is passed through a tortuous sigmoid colon.

FIG. 2 B. Further insertion of the endoscope causes sigmoid loop formation.

FIG. 3 A, B, C, D, E. Examples of most commonly encountered colonic loops:
FIG. 3A—N loop in the sigmoid colon,
FIG. 3B—Alpha loop in the sigmoid colon,
FIG. 3C—reverse Alpha loop in the sigmoid colon,
FIG. 3D—transverse colon loop,
FIG. 3E—transverse colon gamma loop.

FIG. 4 A. Sigmoid loop formed with insertion of the endoscope

FIG. 4 B. The endoscope is partially withdrawn with a torque—the sigmoid colon folds on the shaft of the endoscope.

FIG. 4 C. The endoscope is reintroduced with the clockwise torque; additionally pressure on the abdominal wall is applied manually.

FIG. 5. The example of embodiment of the invention—plastic overtube with longitudinal split, openings and closing mechanisms.

FIG. 6 A. Closing mechanism—opened

FIG. 6 B Closing mechanism—closed

FIG. 6 C. Alternative closing mechanism

FIG. 7 A. Opening on the split.

FIG. 7 B. The endoscope inserted into the overtube protruding via opening.

FIGS. 8 A,B and C. Stages of use of the overtube.
FIG. 8 A Placement of the first segment of the overtube tube on the endoscope,
FIG. 8 B The overtube advanced into the colon on the endoscope.
FIG. 8 C. The endoscope advanced through the overtube.
FIG. 8 D. Further insertion on the segmental overtube and advancement of the endoscope.

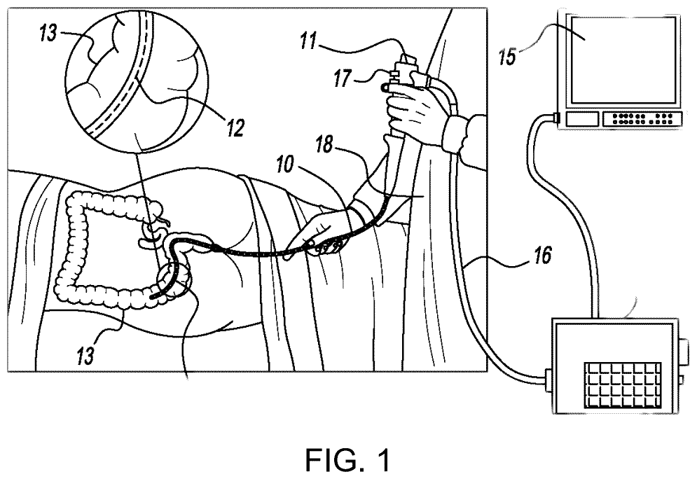

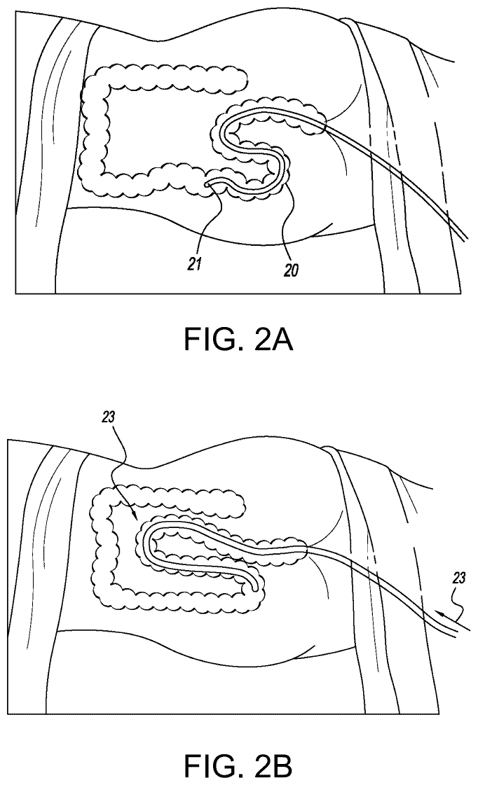

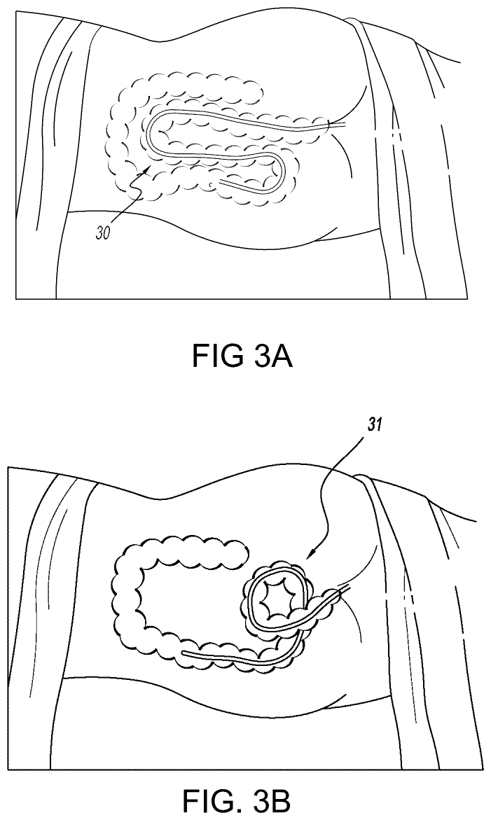

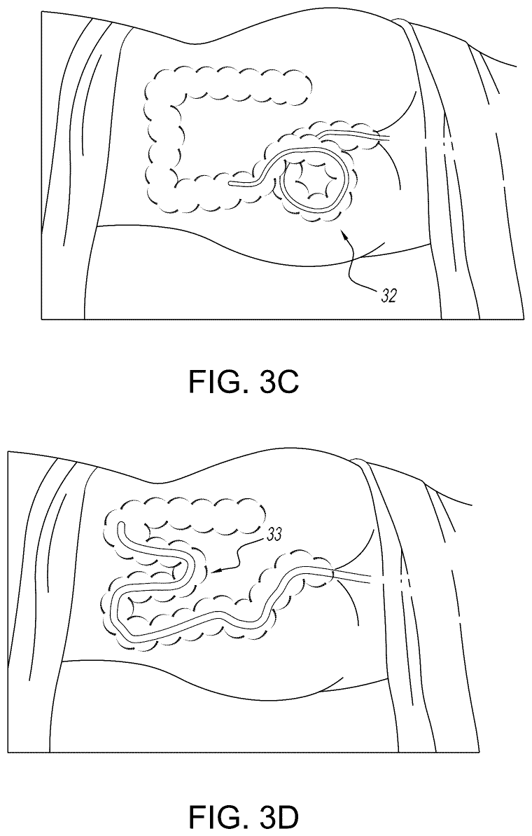

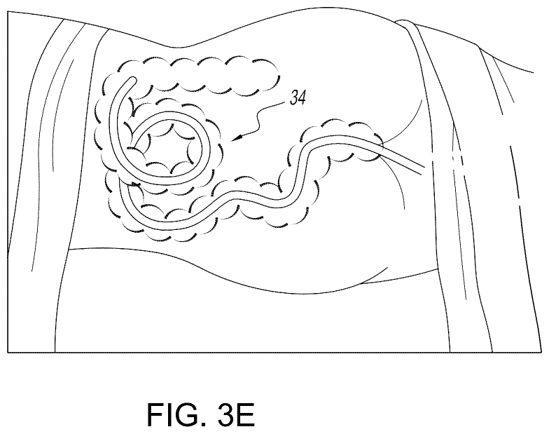

### DETAILED DESCRIPTION

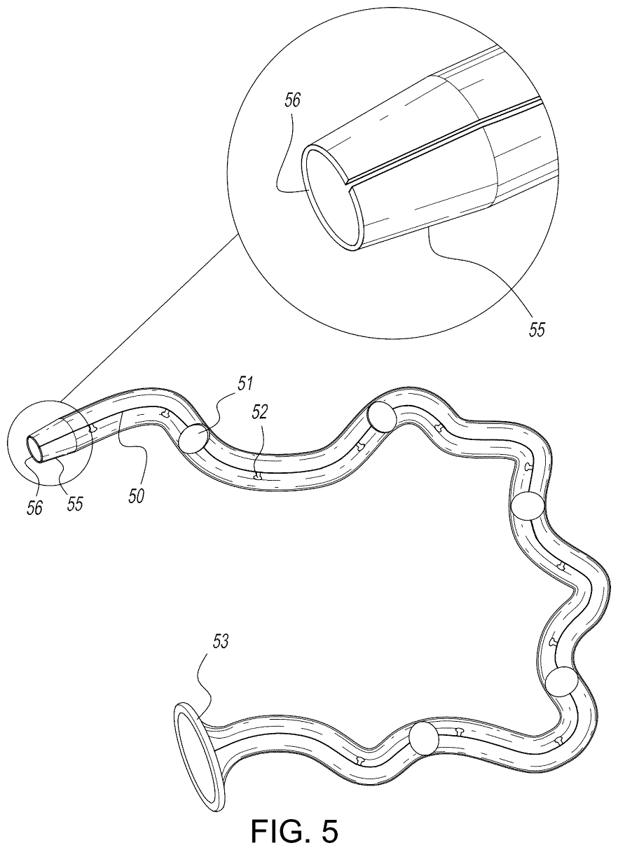

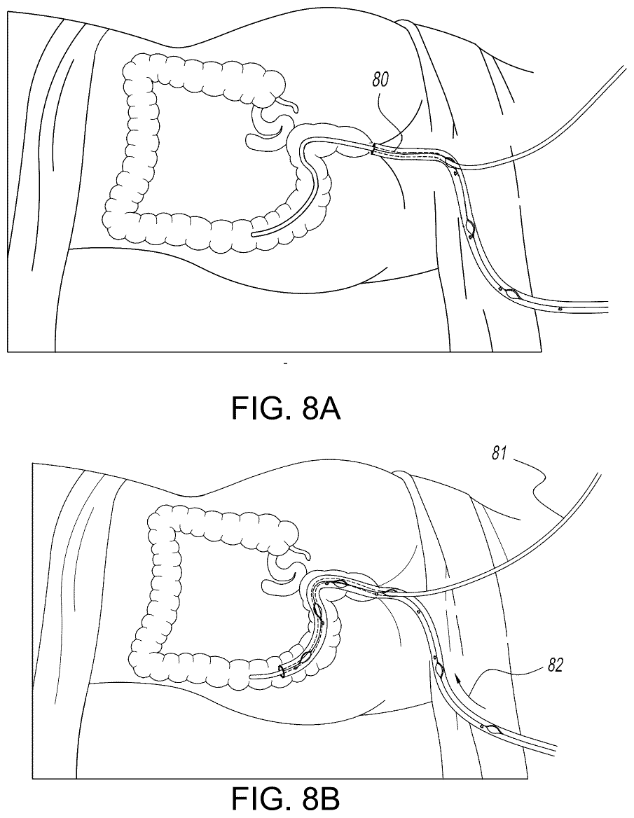

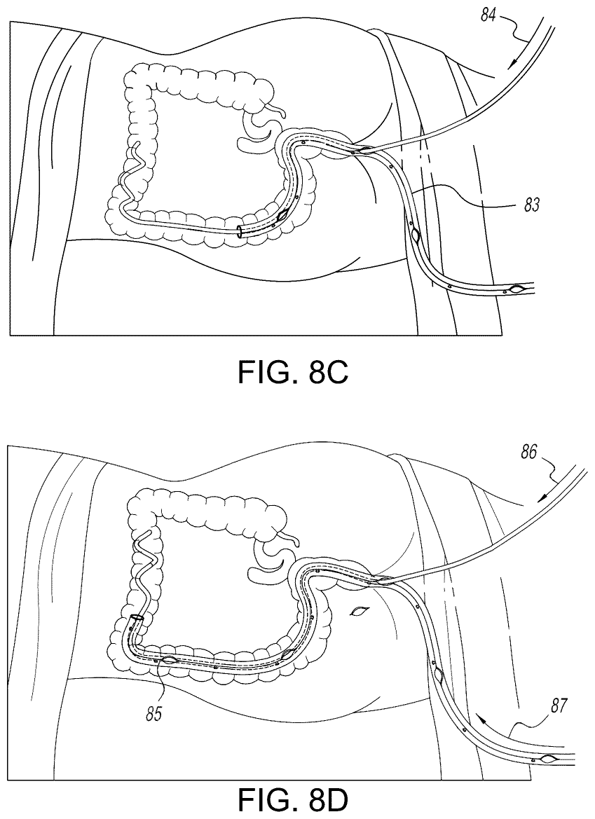

The example of the embodiment of the overtube with longitudinal split is illustrated in FIG. 5. Tube made out of plastic—f.ex. polyurethane, polyethylene, polypropylene, polyamide and polyvinyl chloride—and has thickness of about 1 mm, an internal diameter of 10 mm (0.4 mm above the external diameter of the endoscope) and external diameter of 12 mm. Since colonoscopies are performed using different diameter endoscopes, size specific overtube can be manufactured. The selection of material and its thickness is determined by required stiffness and flexibility. It has to be flexible enough to easily advance as it is pushed in on the endoscope as exemplified on FIG. 8B, yet stiff enough not to allow the endoscope to buckle when the overtube is kept steady as conduit for endoscope—FIG. 8C. The elastic qualities of the material have to be such that once the split FIG. 5—detail 50 is spread out and the endoscope is inserted into the overtube, the overtube easily returns to its original circular shape—FIG. 8A. The overtube can be of variable length—it is generally expected to be between 20 to 60 cm.

The overtube is longitudinally split 50 with straight cut parallel to its axis The split is a straight cut throughout the thickness of the overtube. Other shapes of the split with reference to the overtube axis may be considered. For example, a split can be elongated spiral or wavy.

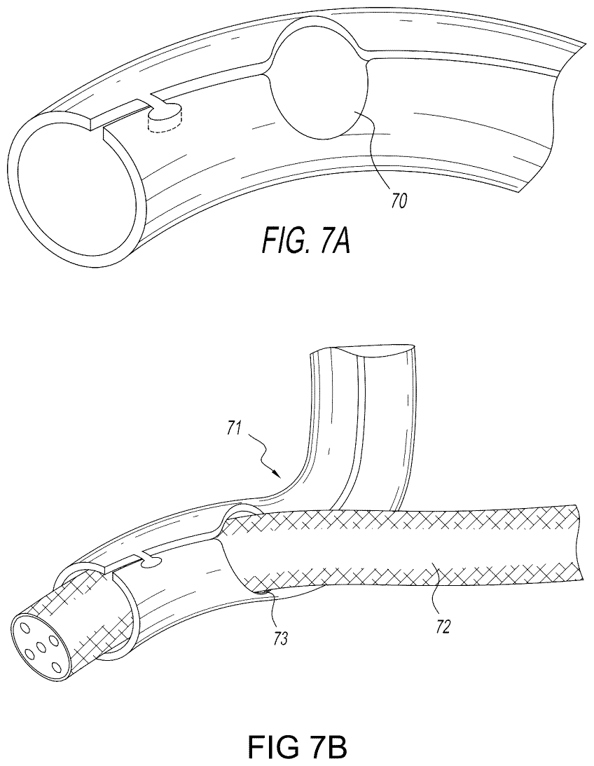

Along the split there may be a plurality of oval openings as illustrated in FIG. 5—detail 51 and FIG. 7A—detail 70. The shape of the opening will be oval with the longest width perpendicular to the axis of the tube approximately 10 mm and the shorter axis, parallel to the axis of the overtube being about 7 mm. The size of the oval opening is such that when the overtube is bent FIG. 7 B—detail 71, it becomes circular and accommodates the shaft of the endoscope 72 without excessive increase in the friction between endoscope and the opening in the overtube 73. The openings of the split are positioned evenly throughout the length of the split every 10 cm. The openings are to facilitate endoscope insertion and sliding within the overtube. Different shapes and positions in relation to the split can be tried f.ex. the openings can be positioned in shorter or longer intervals as well as irregular intervals.

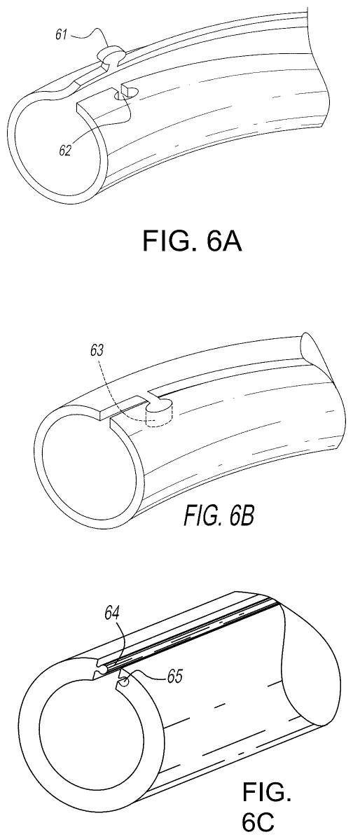

Along the split there are closing mechanisms 52 and FIGS. 6A and 6B consisting of mushroom-like protrusion 61 on one of the edges and corresponding cutout 62 on the other edge are positioned. After the edges of the split are approximate the protrusion fits tightly in the cutout which closes the overtube 63. These closures can be positioned in variable distances along the split, generally in the proximity of the openings and the tip of the overtube. Alternative closing mechanisms may be used as exemplified in FIG. 6C. It consists of a longitudinal ridge with protruding profile 64 in the middle of one edge thickness with corresponding groove 65 in the other edge. It can be closed in the ziplock fashion after the endoscope is placed inside the overtube.

The overtube has uniform diameter throughout its length except for its ends. The end closer to the handle of the endoscope when inserted is called the proximal end. It has gradually flared up to about 4-5 cm in diameter over about 2 cm of the length of the overtube—FIG. 5 detail 53. The purpose of this flared up shape of the proximal end is to prevent accidental insertion of the entire overtube into the colon. The other end, called distal, is tapered 55 over approximately 2 cm with internal diameter approaching the external diameter of the endoscope. It also has rounded edge 56—these features allow the tip of the overtube to closely embrace the shaft of the endoscope and minimize the risk of mucosal injury while introduced into the colon.

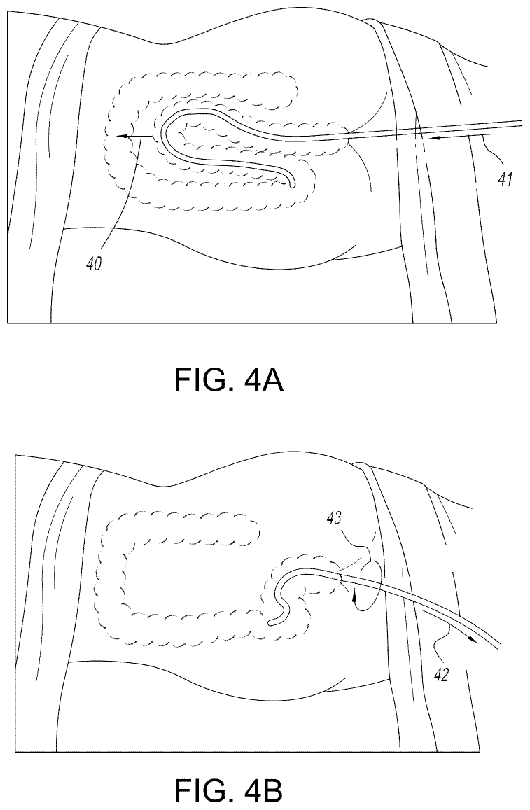

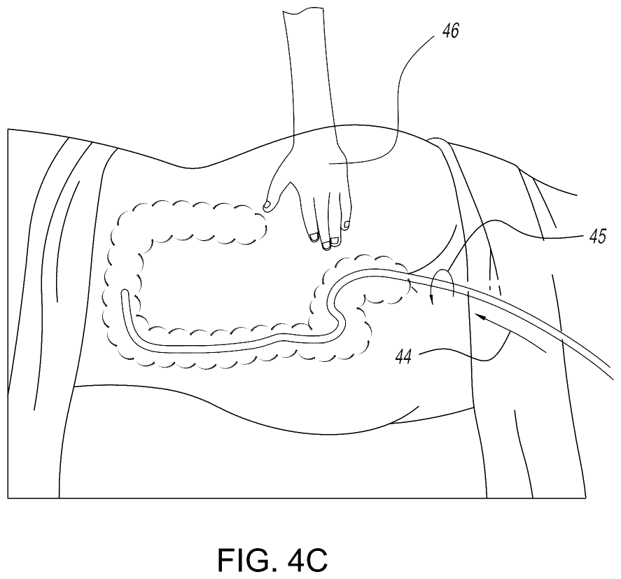

The method of the insertion of the split overtube and its action to prevent looping is schematically represented in FIGS. 8A, 8B, 8C, and 8D. When the operator can not advance the endoscope and suspects loop formation the standard maneuvers are employed—this is described in detail in the background section and illustrated in FIGS. 4A, 4B and 4C. When these measures fail and loop reforms repeatedly the split overtube can be employed. First the endoscope is withdrawn which straightens it. The edges of the most distal segment of the overtube—between the tip and first oval opening—are spread out and the shaft of the endoscope is forced into the overtube lumen just outside of the patient's body FIG. 8A detail 80. Then the closing mechanism (FIG. 6A, B) is engaged near the distal end of the overtube and near the first opening along the split. This additionally assures that the endoscope is fully encompassed inside the overtube.

The overtube is then inserted to the colon 82 by sliding over the endoscope that is held steady 81. The tapered and rounded edge of the distal end of the overtube ensures smooth insertion. The increase in the radius size is negligible and does not impede insertion into the lumen of the colon. Small amount of lubrication on the endoscope shaft may be used to facilitate the process of overtube sliding in. Subsequently additional sections of overtube 82 are used to encompass the shaft of the endoscope and are inserted in similar fashion until desired length of the endoscope inside is covered. Because the looping occurs most commonly in the sigmoid colon it is expected 20-40 cm of the overtube (2-4 sections) will be used in most cases.

Once the overture is inserted, it is bent at the level of first widening visible outside—FIG. 8C. From this point the overtube 83 is kept at a steady position and provides semi-rigid conduit for the endoscope which is then advanced deeper into the colon 84 in the usual manner. These steps of sliding of the overtube over the shaft of the endoscope 87 and advancing the endoscope through the overtube conduit can be repeated if needed as illustrated on FIG. 8D. Once the endoscope reaches the end of the desired insertion site (usually end of colon called cecum) the overtube can be withdrawn. Alternatively it can be left on the endoscope until its tip reaches the distal end of the overtube and it can be withdrawn together with the endoscope.

I claim:

1. An overtube for use with an endoscope during colonoscopy, the overtube comprising a tubular body defining a hollow, elongated structure with substantially uniform internal diameter, having a proximal end and a distal end, and a longitudinal split extending along the length of the tubular body, wherein the edges of the longitudinal split are configured to be temporarily spread apart to accommodate the insertion of the shaft of the endoscope and to resiliently return to their original positions, thereby securing the endoscope shaft within the overtube.

2. The overtube of claim 1, wherein the tubular body is formed from a plastic material having elastic properties sufficient to allow the longitudinal split to spread and return to its original position, the plastic material selected from the group consisting of polyurethane, polyethylene, polypropylene, polyamide, and polyvinyl chloride.

3. The overtube of claim 1, wherein the tubular body is configured to have variable lengths, thereby providing selectable coverage for different portions of the endoscope shaft during use.

4. The overtube of claim 1, wherein the overtube is provided with an internal diameter larger than the external diameter of the endoscope shaft, creating a variable lumen diameter, the overtube sized to accommodate endoscopes of different diameters.

5. The overtube of claim 1, wherein the proximal end of the tubular body is flared out to a diameter at least twice that of the tubular body external diameter, thereby preventing accidental insertion of the entire overtube into the colon.

6. The overtube of claim 1, wherein the distal end of the tubular body is tapered to closely match the external diameter of the endoscope shaft, the distal end further comprising a rounded edge to ensure atraumatic insertion of the overtube into the colon.

7. The overtube of claim 1, wherein the longitudinal split extends in a straight path along the axis of the tubular body, parallel to the long axis of the overtube.

8. The overtube of claim 1, wherein the longitudinal split is configured in a nonlinear path, selected from a group consisting of spiral, wavy, or zigzag patterns.

9. The overtube of claim 1, further comprising a plurality of oval-shaped openings disposed along the length of the longitudinal split, the openings configured to facilitate bending of the overtube in order to provide easier exit of the endoscope from the lumen of the overtube.

10. The openings of claim 9, wherein the oval-shaped openings are positioned at variable intervals along the length of the longitudinal split to optimize overtube flexibility and ease of use.

11. The overtube of claim 1, wherein the longitudinal split is further provided with a closing mechanism disposed in proximity to the distal end and each of the oval openings, the closing mechanism operating to close and secure the edges of the split after the endoscope shaft has been inserted into the lumen of the overtube.

12. The closing mechanism of claim 11, wherein the closing mechanism comprises a mushroom-like protrusion on one edge of the split and a corresponding cutout on the opposite edge, the protrusion engaging the cutout to hold the edges of the split in a closed position.

13. The closing mechanism of claim 11, wherein the closing mechanism comprises a longitudinal ridge-and-groove system, the ridge extending along one edge of the split and configured to engage a corresponding groove in a zip-lock fashion.

14. A method of using the overtube of claim 1, the method comprising:
(a) temporarily spreading the edges of the longitudinal split to accommodate the insertion of the endoscope shaft;
(b) sliding the overtube along the endoscope shaft until the desired length of the overtube is positioned within the colon; and
(c) holding the overtube in place to provide a semi-rigid conduit for the endoscope, thereby enabling the axial force exerted by the operator to be transmitted into forward movement of the endoscope tip, preventing loop formation.
